(19) United States
(12) Patent Application Publication
(10) Pub. No.: US 2005/0261549 A1
(43) Pub. Date: Nov. 24, 2005

(54) Body cavity inspection system and method of using the same

(76) Inventors: James Robert Hewit, Dundee (GB); Alan Peter Slade, Dundee (GB)

Correspondence Address: GIFFORD, KRASS, GROH, SPRINKLE & CITKOWSKI, P.C, PO BOX 7021, TROY, MI, 48007-7021, US

(21) Appl. No.: 10/529,083

(22) PCT Filed: Sep. 23, 2003
(86) PCT No.: PCT/GB03/04144

§ 371 Date: Jun. 27, 2005

(30) Foreign Application Priority Data
Sep. 24, 2002 (GB) 0222106.7

(51) Int. Cl. A61B 1/01

(52) U.S. Cl. 600/114; 600/144; 600/115

(57) ABSTRACT

Transportation apparatus for transportation of an inspection device within a body cavity such as the colon and inspection apparatus for inspecting a body cavity is disclosed. In one embodiment, there is disclosed transportation apparatus (10) for transportation of an inspection device (12) within a body cavity (14), the apparatus comprising: a carrier (16) for insertion into an opening (20) of the body cavity (14), the carrier (16) moveable between a collapsed position and an extended position where the carrier (16) extends along a length of the cavity (14); and a guide member (18) coupled to the carrier (16), the guide member (18) adapted to be carried into the cavity by the carrier (16) when the carrier (16) is moved to the extended position, such that the guide member (18) acts as a guide for transportation of the inspection device (12) within the cavity (14).

The present invention relates to body cavity inspection. In particular, but not exclusively, the present invention relates to transportation apparatus for transportation of an inspection device within a body cavity such as the colon and inspection apparatus for inspecting a body cavity.

There are compelling medical reasons for inspecting body cavities, especially the inner walls of the colon. Such inspection can lead to early detection of cancerous lesions and it is well known that the earlier these are detected, the better is the chance of successful treatment.

Minimal Access Surgery (MAS) is particularly important and widely used for diagnosis and surgery in the gastrointestinal tract. An increasing number of diagnostic interventions are performed by colonoscopy, which involves examination of the colon (the large intestine) using an endoscope. Colonoscopy has become important since cancer of the colon and rectum are the second most malignant forms of tumour in industrialised countries, and in around 90% of cases colon carcinomas, which are malignant growths, develop from a benign internal polyp.

In operations relatively near to the anus, a rigid endoscope may be used. However, to permit operations further along the intestine, more flexible endoscopes, such as fiberoptic endoscopes, are required. These are inserted anally and pushed along the bowel and intestine. A fibreoptic bundle transmits light from a light source to the target area, and images are transmitted back through another fibreoptic bundle, which is arranged in a coherent or parallel manner.

An alternative endoscope is the micro-sensor endoscope, which is also flexible. In a micro-sensor endoscope, the optical elements are replaced by micro-sensors and electronic wiring. With adequate light from a non-coherent fiberoptic bundle, video images are created for storage as well as for viewing in a diagnosis procedure using a monitor. Such a viewing system is called an indirect video system and typically has 25% less resolution compared with a direct video system such as a fiberoptic endoscope.

Comparative studies show that flexible endoscopes detect an average of three times as many polyps and cancers as do rigid endoscopes. Although, fiberoptic and micro-sensor endoscopes are more flexible than conventional rigid endoscopes, their stems, which are made of long rubber or plastic tubes, are positionally uncontrollable over most of their length. This is a problem because the human colon is composed of a set of labyrinthine and reverse bends, the five major parts of the colon comprising the rectum, the sigmoid, descending, transverse, and ascending colon. The smallest radius of curvature is approximately 2-3 cm, found at the bending portion between the rectum and the sigmoid colon. The transverse colon, which is 40-50 cm long, is the largest and most mobile part of the colon and extends between the right and left colic flexures, forming a loop that is directed downwards and forwards.

As the transverse colon is suspended posteriorly by the soft living tissue known as the transverse mesocolon, its movements are always affected by the breathing process and other movements in the intestinal cavity. In particular, use of a colonoscope is impeded by peristaltic action of the gastrointestinal (GI) tract, which is continuously attempting to expel the device. Involuntary motions of the GI tract create difficulties in acquiring a target and in using the array of diagnostic and therapeutic tools that are deployed through a channel in the stern. The GI tract may also cramp, thus trapping the device inside the colon. High mechanical flexibility is therefore required for the endoscope to traverse the colon without creating potentially damaging interacting forces.

However, the very compliance required in the endoscope stem makes maneuvering the endoscope around the bends of the colon extremely difficult. So called alpha (α) loops are often created by the endoscopist to help advance the stem at, for example, the reverse bends, junctions of the sigmoid/descending colon and the descending/transverse colon. Twisting and retracting of the stem is often required to make these loops, hence high interacting forces between the endoscope and the wall of the colon are inevitable.

Steering systems at the tip of the endoscope provide two directional controls which are up-down and left-right, manually operated using two control knobs. In this respect, flexible endoscopes can be considered in the class of teleoperated manipulators, with limited degrees of freedom and direct mechanical master-slave coupling. This manual procedure, which bears no particular relation to the resulting motions, has the potential to damage the surrounding tissue. Successful operation and manipulation of these medical devices also requires great proficiency as well as a great deal of time. These are skills which need to be mastered by endoscopists worldwide. Despite this, the length of flexible endoscopes is still limited to approximately 1 m, which leaves 80% of the digestive tract unexplored.

Some researchers have proposed bowel climbing robotic devices to carry miniature cameras through the bowel to the colon. One particular embodiment of this concept is the “inchworm” robot developed by the University of Pisa (Italy), which consists of an extendable body mounted between two cylindrical pistons. These pistons have variable diameters which may be changed by internal actuation allowing the robot to “walk” along the colon. The walking process is carried out by first extending the rear piston to full diameter, engaging the bowel wall. Next, the front piston is retracted and disengages from the wall. The body is then extended, driving the front piston forward. The front piston is then again extended and engages the wall. Finally, the rear piston retracts and the body shrinks, pulling the rear piston forward. This “inching” movement is repeated as necessary.

This kind of robot, like all others that use the bowel wall for leverage, present risks of harming the patient by damaging or rupturing the bowel wall. The robot is also slow and may be difficult to extricate in the event of power failure. This problem is typical of those found in many proposals to use robots to aid in surgical procedures and is based on the assumption that a surgical robot is simply an industrial robot in unfamiliar surroundings. Indeed, the Pisa robot is to an extent a simple modification of the familiar pipe-traversing “pig”, well known in the oil and gas industries.

Various alternative devices for insertion into body cavities are disclosed in W096/01130 (Origin Medsystems); W087/05523 (Siemens); W085/00097 (Sterimed); US 2002/0016607 (Bonadio et al); U.S. Pat. No. 6,485,409 (Voloshin et al); U.S. Pat. No. 4,321,915 (Leighton et al); and U.S. Pat. No. 3,525,329 (Zeimer).

It is amongst the objects of embodiments of the present invention to obviate or mitigate at least one of the foregoing disadvantages.

According to a first aspect of the present invention, there is provided a carrier for facilitating transport of a member into a body cavity, the carrier being adapted for insertion into an opening of the body cavity, the carrier moveable between a collapsed position and an extended position where the carrier extends along a length of the body cavity.

According to a second aspect of the present invention, there is provided transportation apparatus for transportation of an inspection device within a body cavity, the apparatus comprising:

a carrier for insertion into an opening of the body cavity, the carrier moveable between a collapsed position and an extended position where the carrier extends along a length of the cavity; and

a guide member coupled to the carrier, the guide member adapted to be carried into the cavity by the carrier when the carrier is moved to the extended position, such that the guide member acts as a guide for transportation of the inspection device within the cavity.

According to a third aspect of the present invention, there is provided inspection apparatus for inspecting a body cavity, the apparatus comprising:

a carrier for insertion into an opening of the body cavity, the carrier moveable between a collapsed position and an extended position where the carrier extends along a length of the cavity;

a guide member coupled to the carrier, the guide member adapted to be carried into the cavity by the carrier when the carrier is moved to the extended position; and

an inspection device adapted to co-operate with the guide member for transportation within the cavity.

The invention allows an inspection device to be easily and quickly inserted into a body cavity to allow an inspection procedure to be carried out. Also, the invention avoids damage to the body cavity during insertion and subsequent use. Furthermore, the invention allows safe inspection along a relatively large portion of body cavities, in particular, the colon, not possible with prior art apparatus.

The inspection device may be adapted to be transported within the cavity along the guide member. Alternatively, the inspection device may be coupled to the guide member and may be adapted to be transported within the cavity by the guide member. Thus, in embodiments of the invention, the inspection device may be drawn through the cavity by retracting the guide member along the cavity towards the cavity opening. The guide member may comprise an elongate coupling connected to the inspection device, which may allow transmission of data from the inspection device and may couple the device to a source of power. The inspection device may be coupled to the guide member such that in the carrier extended position, the inspection device is located within the carrier, or externally of the carrier.

Preferably, the guide member is moveable between a relaxed state or configuration and a rigid state or configuration, the guide member acting as a guide when in the rigid state. It will be understood that, in the rigid state, the guide member may be sufficiently rigid to allow transportation of the inspection device within the cavity, whilst remaining sufficiently flexible so as not to damage the walls of the cavity. The guide member may be adapted to be carried into the cavity in the relaxed state. Accordingly, the guide member may be adapted to be moved to the rigid state following movement of the carrier to the extended position. Thus, the guide member may initially be in the relaxed state to aid insertion of the guide member into the cavity using the carrier.

Preferably also, the guide member is reversibly moveable or switchable between the rigid state and the relaxed state, and may be selectively reversibly moveable between said states. This allows the guide member to be returned to the relaxed state, assisting in removal of the guide member from the body cavity. The guide member may also be lockable. Thus, the guide member may be lockable in the rigid state.

Alternatively, the guide member may be at least partly rigid. This may allow a force to be transmitted to the guide member to assist insertion of the guide member into the cavity using the carrier. The guide member may comprise an endoscope such as a colonoscope.

Preferably also, the guide member is releaseably coupled to the carrier. This allows the carrier to be released from the guide member and removed from the cavity after the guide member has been located in the cavity. The apparatus may further comprise a releaseable coupling for releaseably coupling the carrier to the guide member. The releaseable coupling may be adapted to be actuated to release the carrier from the guide member. The coupling may comprise a shape memory alloy (SMA) coupling and may comprise a loop or tie coupled to the carrier.

Alternatively, the carrier may be adapted to remain coupled to the guide member during transportation of the inspection member within the cavity. The carrier may thus be of a nature (for example, of a low friction, and/or translucent material) to allow an inspection procedure to be carried out without removing the carrier from the cavity, or the carrier may be releaseable from the guide member following location of the inspection device at a desired position in the cavity.

The guide member may define a plurality of engagement portions adapted to be engaged by the inspection device for transporting the device within the cavity. The guide member may comprise a plurality of engagement portions coupled together to form the guide member. Alternatively, the guide member may comprise an elongate support such as wire, cable or tube with a plurality of engagement portions mounted on the support. The engagement portions may comprise bodies defining an engagement surface, a protrusion such as a tooth, a recess or any other shaped surface. The guide member may be adapted to be moved to the rigid state by tensioning the elongate support. Thus the engagement portions may be moveably mounted on the elongate support.

Alternatively, the guide member includes a plurality of locking elements for locking the guide member. The locking elements may be actuated to move the guide member to the rigid state. The locking elements may comprise shape memory alloy (SMA) locks or switches or any alternative electrically actuated locking elements. In embodiments of the invention, the locking elements may comprise a first set of locking elements adapted to be actuated to rigidise the guide member and a second set of locking elements adapted to be actuated to relax the guide member. The first and second sets of locking elements may be adapted to be actuated at different temperatures. Thus, the first set may be adapted to be actuated at a first temperature to rigidise the guide member and the second set may be adapted to be actuated at a second, lower temperature. Preferably, at least part of the guide member is electrically conductive to allow an electric current to be passed along the guide member, for actuation of the locking elements. Accordingly, the guide member can easily be moved between the relaxed and rigid states by locking and unlocking the locking elements. The electric current may generate heat for moving the guide member between relaxed and rigid states.

The inspection device may be adapted to engage the guide member for transportation within the cavity. The inspection device may be self-driven and may include a drive mechanism for engaging the guide member. Alternatively, or additionally, the inspection device may be adapted to be externally driven. The guide member may be externally driven by a tube or other semi-rigid member mounted on the carrier. The drive mechanism may act as a follower mechanism if the inspection device is externally driven. The drive mechanism may comprise teeth for engaging engagement portions of the guide member.

Preferably, the carrier is flexible when in the collapsed position and adapted to be constrained by a wall of the body cavity when in the extended position. The carrier may have sufficient strength such that, during movement to the extended position, the carrier is able to carry the guide member into the cavity and to support the guide member, whilst remaining sufficiently flexible so as to follow the path of the body cavity without damaging the walls of the cavity. Most preferably, the carrier is inflatable and may comprise an inflatable bag such as an elongate balloon or closed-end tube. The carrier may therefore be simply extended by supplying a gas under pressure to the carrier. The carrier may be adapted to be inserted into the opening of the body cavity in an everted (inside-out) position.

According to a fourth aspect of the present invention, there is provided a method of transporting an inspection device within a body cavity, the method comprising the steps of:

coupling a guide member for the inspection device to a flexible carrier;

inserting the flexible carrier into an opening of the cavity in a collapsed position and moving the carrier to an extended position where the carrier extends along a length of the cavity, the carrier carrying the guide member into the cavity during movement to the extended position; and

transporting the inspection device within the cavity using the guide member.

The guide member may be carried into the cavity in a relaxed state and subsequently moved to a rigid state. The guide member may be releaseably coupled to the carrier and following movement of the carrier to the extended position, the carrier may be released from the guide member and recovered from the cavity. Alternatively, the carrier may remain coupled to the guide member during transportation of the inspection device within the cavity. Preferably, the guide member may be moved from the rigid state to the relaxed state to allow removal of the guide member from the cavity.

Preferably, the carrier is inflatable to move to the extended position. The carrier may be inflated by supplying a pressurised gas to the carrier.

Preferably, the inspection device is transported within the cavity along the guide member. The inspection device may be self-driven along the guide member and may be remotely controlled. This allows an operator to control movement of the inspection device within the body cavity. Alternatively, the inspection device may be externally driven. For example, a drive member such as a tube may be coupled to the inspection device for transporting the inspection device along the guide member.

Alternatively, the inspection device may be coupled to the guide member and may be drawn through the cavity by the guide member. The inspection device may be coupled to the guide member such that when the carrier is extended, the inspection device is located within the carrier, or externally of the carrier. The inspection device may be transported to an end of the guide member located within the cavity and the guide member may then be retracted from the body cavity. Thus the inspection device may be transported back through the cavity by the guide member, allowing inspection of the cavity during this movement. Preferably, the guide member is moved to the relaxed state prior to retraction from the body cavity.

According to a fifth aspect of the present invention, there is provided a carrier for insertion into an opening of a body cavity, the carrier moveable between a collapsed position and an extended position where the carrier extends along a length of the body cavity, for carrying an endoscope into the body cavity.

According to a sixth aspect of the present invention, there is provided inspection apparatus for inspecting a body cavity, the apparatus comprising:

a carrier for insertion into an opening of the body cavity, the carrier moveable between a collapsed position and an extended position where the carrier extends along a length of the cavity; and

an inspection device coupled to the carrier.

Embodiments of the present invention will now be described, by way of example only, with reference to the accompanying drawings, in which:

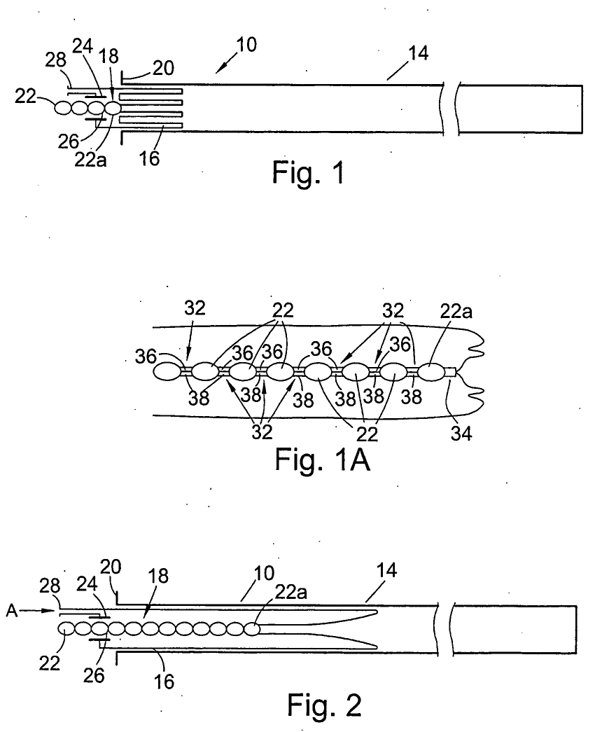

FIG. 1 is a schematic, partial cross-sectional view of transportation apparatus forming part of inspection apparatus in accordance with a preferred embodiment of the present invention, illustrating a first step in a procedure for inserting the transportation apparatus into a body cavity;

FIG. 1A is an enlarged view of the apparatus of FIG. 1;

FIG. 2 is a view of the apparatus of FIG. 1 shown part way through the procedure for inserting the transportation apparatus into the body cavity;

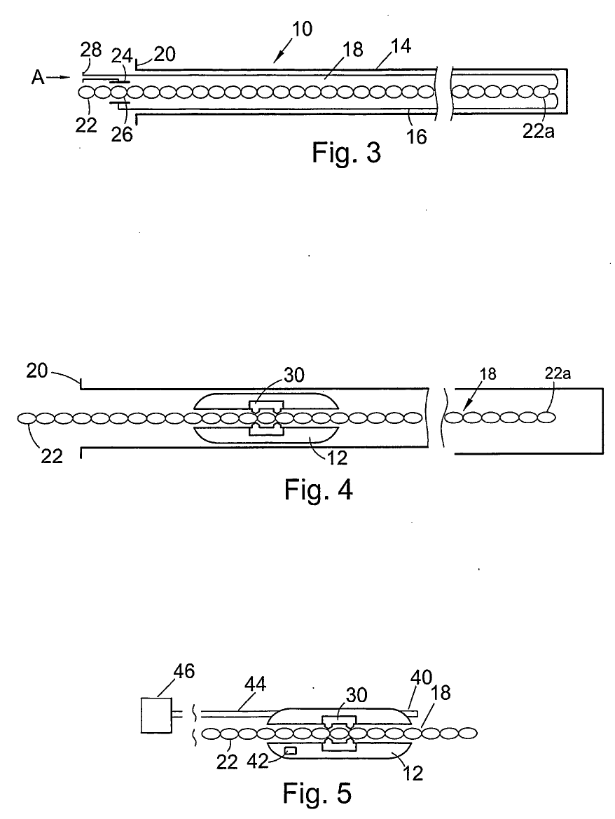

FIG. 3 is a view of the apparatus of FIG. 1 illustrating the transportation apparatus following full insertion into the body cavity;

FIG. 4 is a view of the apparatus of FIG. 1 following removal of a carrier of the apparatus from the body cavity and during transportation of an inspection device within the cavity using the transportation device;

FIG. 5 is an enlarged view of the apparatus shown in FIG. 4;

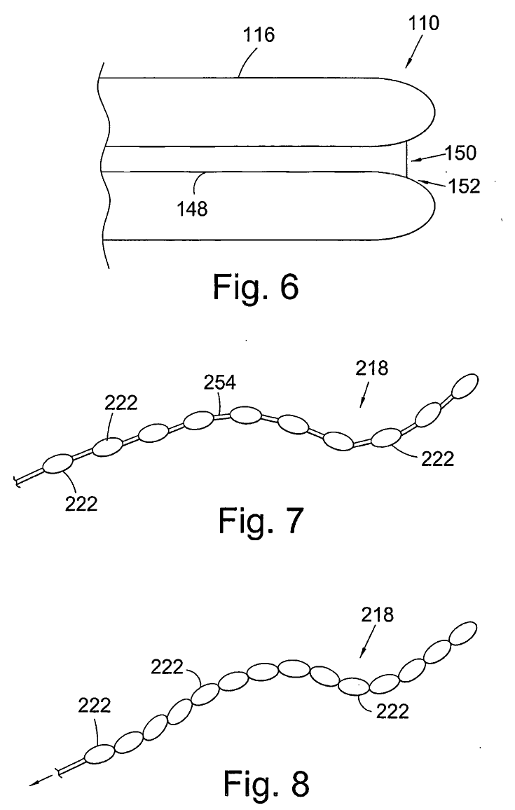

FIG. 6 is a schematic, partial cross-sectional view of transportation apparatus forming part of inspection apparatus in accordance with an alternative embodiment of the present invention; and

FIGS. 7 and 8 are views of a guide member forming part of alternative transportation apparatus, shown in flexible and rigid configurations, respectively.

Turning firstly to FIG. 1, there is shown transportation apparatus indicated general by reference numeral 10, forming part of an inspection apparatus for inspecting a body cavity. The inspection apparatus includes the transportation apparatus 10 and an inspection device in the form of an inspection robot 12 shown in FIG. 4, which will be described below.

The transportation apparatus 10 is used for transporting the inspection robot 12 within the body cavity, which in this example comprises the colon 14 of a patient. The transportation apparatus 10 includes a carrier in the form of an inflatable balloon 16 and a guide member in the form of a robot ladder 18.

The balloon 16 is shown in FIG. 1 in an everted, collapsed position where the balloon has been inserted into an opening, the anus 20 of the colon 14 of a patient. The robot ladder 18 is coupled to the balloon 16, and the balloon 16 is moved to an extended position where the balloon extends along a length of the colon 14. As the balloon 16 is extended, the robot ladder 18 is also carried into the colon 14. In this position, the robot ladder 18 acts as a guide for transportation of the inspection robot 12 within the colon 14. Damage to the bowel wall is thus prevented by the balloon 16, which provides a protective covering for the wall, and by the fact that there is no relative movement between the balloon 16 and the wall as it unrolls along the inside.

In more detail and as shown in the enlarged view of FIG. 1A, the robot ladder 18 comprises a plurality of engagement bodies 22 which are each pivotally coupled together to form the ladder 18. A number of locking elements in the form of shape memory alloy (SMA) locks are coupled between the bodies 22 and are actuated to move the ladder 18 to a rigid state. A leading body 22a of the ladder 18 is releaseably coupled to the balloon 16 and the ladder 18 extends through a seal 24 which is mounted in a ladder opening 26 of the balloon, to reduce leakage from the balloon 16 when inflated. The balloon 16 also includes an inflation opening 28 through which a pressurised gas may be pumped into the balloon 16 by a compressor (not shown), to inflate the balloon and move the balloon to the extended position.

Following insertion of the balloon 16 into the anus 20, the pressurised gas, preferably an inert gas such as carbon dioxide, is pumped into the balloon through the opening 28, as indicated by the arrow A in FIG. 2. This begins to inflate the balloon 16 which then expands and extends from the collapsed position, travelling along a length of the colon 14. As the balloon 16 extends, the ladder 18, which is in the relaxed state, is carried into the colon 14. It will be understood that, for ease of illustration, the colon 14 is shown as a straight passage. However, as described above, the colon is in fact a complex twisting structure.

Inflation of the balloon 16 continues until the balloon is fully expanded, as shown in FIG. 3, indicated to the operator by an increase in the pressure of the gas, measured by suitable gauges in the compressor. The opening 28 is then either sealed, or gas is continued to be pumped into the balloon 16 to maintain the balloon in the inflated, extended position, to account for gas escape through the seal 24. When it has been verified that the balloon 16 is fully extended, and thus that the ladder 18 has been carried fully into the colon 14, the balloon 16 is released from the ladder 18.

The balloon 16 is released by passing an electrical current along the ladder 18, to heat an integral electric heating element (not shown). This causes an SMA lock, tie or loop 34 (FIG. 1A) attaching the body 22a to the balloon 16 to release the balloon, allowing retraction of the balloon 16 from the colon 14 through the anus 20. Simultaneously, the current causes each of the SMA locks to be actuated to move the ladder 18 to the rigid state, allowing transportation of the robot 12 within the colon 14. It will be understood that the SMA locks undergo a phase transformation in their crystal structure when heated, from a weaker deformable structure to a stronger, high temperature structure in which the SMA exhibits superelastic properties.

The apparatus 10 actually includes a first set of locks 36 which are moved to a deformed position to move the ladder 18 to the rigid state and a second set of locks 38 (both shown in FIG. 1A) back-to-back with the first set, for moving the ladder 18 to the relaxed state. As will be described, the first and second sets of locks 36, 38 have different transformation temperatures. The ladder 18 is in the relaxed state for insertion into the colon 14. When a current is passed through the ladder 18, this causes the first set of SMA locks 36 to become heated to above their transformation temperature. The locks 36 thus undergo phase transformation, becoming superelastic and returning to their undeformed position, rigidising the ladder. To move the ladder 18 to the relaxed state, the current is switched off, cooling the SMA locks 36, 38. The second set of locks 38 then return to their undeformed position, at a lower transformation temperature, returning the ladder 18 to the relaxed state.

In the rigid state, the engagement bodies 22 of the ladder 18 may be engaged by a drive mechanism 30 of the robot 12, shown in FIG. 4 enabling the robot 12 to travel along the ladder 18 within the colon 14. The robot 12 is equipped with a camera system 40 shown in the enlarged view of FIG. 5 for inspection of the colon 14 during passage through the colon. The robot 12 is powered either by an internal battery 42 or through a power/control umbilical connection 44 extending from the robot 12 to a control system 46 external of the colon and anus, which allows control of the movement of the robot 12. Accordingly, the robot 12 may be instructed to move in a desired direction along the ladder 18 by the operator. This also allows control of the camera system 40, which may for example be rotated or zoomed in or out to obtain a desired image.

The robot is driven to the far end of the ladder 18 (adjacent body 22a) and the electrical current passing through the ladder 18 is switched off. This causes the ladder 18 to return to the relaxed state, allowing removal of the ladder 18 from the colon 14. The ladder 18 is then pulled slowly back out of the colon 14, carrying the robot 12. As the robot is pulled through the colon 14 to the anus 20, images taken by the robot camera system 40 are viewed by the operator. The ladder 18 and robot 12 are then extracted through the anus 20. The inspection process may therefore comprise simply driving the robot 12 to the end of the ladder 18 and viewing the colon 14 on retraction of the ladder.

Alternatively, the colon 14 may be inspected both during movement of the robot 12 to the far end of the ladder 18, and during movement of the robot 12 back along the ladder 18 to the anus 20. The robot 12 is then extracted and the electrical current passing through the ladder 18 is switched off, causing the ladder 18 to return to the relaxed state, allowing removal of the ladder 18 from the colon 14. In a further alternative, the robot 12 may be driven to a location part way along the ladder 18, and then drawn through the cavity by the ladder, as described above. This allows the robot 12 to be driven along the ladder 18, if desired, for example, to conduct a second examination of an area of the colon 14 initially viewed when the robot 12 is retracted using the ladder 18. This is achieved by driving the robot 12 back a distance along the ladder 18.

It will be understood that if no current is applied to the ladder 18, the ladder remains flexible, whereas when current is applied, the ladder becomes rigid. Thus the system is inherently fail-safe, as in the event of malfunction or power loss, the ladder 18 will automatically become flexible to allow retraction.

Data obtained by the robot is analysed during movement of the robot 12 within the colon (real-time), to determine the location of any polyps, tumours or other abnormalities in a diagnostic procedure. This allows treatment such as a surgical procedure to be carried out simultaneously by the robot itself, or subsequently to removal of the robot 12 from the colon 14. Images obtained may also be recorded for further subsequent analysis, for example, to determine the relative location of a polyp such that successful treatment may be verified in a subsequent inspection procedure at a later date.

Turning now to FIG. 6, there is shown a schematic, partial cross-sectional view of transportation apparatus forming part of inspection apparatus in accordance with an alternative embodiment of the present invention, indicated generally by reference numeral 110. Like components of the apparatus 110 with the apparatus 10 of FIGS. 1 to 5 share the same reference numerals incremented by 100.

The apparatus 110 includes a carrier 116 which may be used to assist in the insertion of a conventional, flexible endoscope 148. For example, the endoscope 148, which may be a colonoscope, is coupled to the carrier 116 by coupling an inspection end 150 of the colonoscope 148 to a closed end 152 of the carrier 116. As the carrier 116 is extended, the colonosope 148 is pulled up through the colon, the pulling force of the carrier 116 augmenting a pushing force exerted on the colonoscope 148. The carrier 116 thus protects the bowel wall from damage, resulting in a faster, less traumatic and less dangerous means of insertion of the colonoscope.

Turning to FIG. 7, there are shown views of a guide member 218 forming part of alternative transportation apparatus, shown in flexible and rigid configurations, respectively. Like components of the guide member 218 with the guide member 18 shown in FIGS. 1 to 5 share the same reference numerals, incremented by 200.

The guide member 218 takes the form of a robot ladder and includes engagement bodies 222 movably mounted on an elongate tensionable support 254. The ladder 218 is shown in FIG. 7 in a relaxed, flexible insertion configuration and in FIG. 8 in a rigid, tensioned configuration. The ladder 218 is inserted in the flexible configuration using a carrier (not shown), as described above, and the support 254 is then tensioned to shorten the distance between the bodies 222, rigidising the ladder 218 for passage of a robot (not shown) along the ladder.

Various modifications may be made to the foregoing within the scope of the present invention.

For example, the inspection device may be provided with various further systems for carrying out desired procedures within the body cavity. For example, the inspection device may include apparatus for carrying out a surgical or diagnostic procedure, such as removal of a polyp or tumour or extraction of a portion of the polyp or tumour for subsequent analysis.

The inspection device may be externally driven for transportation within the body cavity. For example, a tube may be mounted on the guide member and connected to the inspection device for manually moving the robot. The tube may be semi-rigid to enable a force to be exerted on the inspection device to move it within the body cavity, the tube being guided by the guide member. The tube may be flexible so as not to damage the body cavity, but possessing sufficient strength to allow transmission of a push/pull force to the inspection device.

The guide member may be moved to a rigid state by any alternative suitable method, for example, the guide member may include micro switches (for example, in place of elements 32) which may move to a position where they restrain movement of an engagement body of the guide member relative to an adjacent body, to rigidise the guide member. The robot 12 may alternatively comprise a follower mechanism enabling external driven movement of the robot along the ladder. In a further alternative, the drive mechanism 30 of the robot 12 may act as a follower mechanism to allow external driven movement of the robot. This may allow recovery of the robot in the event of a power failure.

The inspection device may include a cutting element for separating the carrier from the guide member (for example, in place of loop 34). The device may therefore be driven to an end of the guide member and used to cut the carrier, which is then retracted from the cavity.

1-48. (canceled)

49. Transportation apparatus for transportation of an inspection device within a body cavity, the apparatus comprising:
a carrier for insertion into an opening of the body cavity, the carrier moveable between a collapsed position and an extended position where the carrier extends along a length of the cavity; and
a guide member coupled to the carrier, the guide member adapted to be carried into the cavity by the carrier when the carrier is moved to the extended position, such that the guide member acts as a guide for transportation of the inspection device within the cavity.

50. Apparatus as claimed in claim 49, wherein the guide member is moveable between a relaxed state and a rigid state, the guide member acting as a guide when in the rigid state.

51. Apparatus as claimed in claim 50, wherein the guide member is adapted to be carried into the cavity in the relaxed state and adapted to be moved to the rigid state following movement of the carrier to the extended position.

52. Apparatus as claimed in claim 50 wherein the guide member is reversibly moveable between the rigid state and the relaxed state.

53. Apparatus as claimed in claim 50 wherein the guide member is lockable in the rigid state.

54. Apparatus as claimed in claim 49, wherein the guide member is at least partly rigid.

55. Apparatus as claimed in claim 54, wherein the guide member comprises an endoscope.

56. Apparatus as claimed in claim 49, wherein the guide member is releasably coupled to the carrier.

57. Apparatus as claimed in claim 56, further comprising a releasable coupling for releasably coupling the guide member to the carrier.

58. Apparatus as claimed in claim 57, wherein the coupling comprises a shape memory alloy (SMA) coupling.

59. Apparatus as claimed in claim 49, wherein the guide member defines a plurality of engagement portions adapted to be engaged by an inspection device for transportation of the device within the cavity.

60. Apparatus as claimed in claim 58, wherein the guide member comprises an elongate support with a plurality of engagement portions mounted on the support.

61. Apparatus as claimed in claim 60 when dependent on claim 2, wherein the engagement portions are movably mounted on the elongate support and wherein the elongate support is tensionable to move the guide member to the rigid state.

62. Apparatus as claimed in claim 59 wherein the engagement portions comprise bodies defining an engagement surface.

63. Apparatus as claimed in claim 49 wherein the guide member includes a plurality of locking elements for locking the guide member in a rigid state.

64. Apparatus as claimed in claim 63, wherein the locking elements comprise shape memory alloy (SMA) locks.

65. Apparatus as claimed in claim 63 wherein the locking elements comprise a first set of locking elements adapted to be actuated to rigidise the guide member and a second set of locking elements adapted to be actuated to relax the guide member.

66. Apparatus as claimed in claim 65, wherein the first and second sets of locking elements are adapted to be actuated at different temperatures.

67. Apparatus as claimed in claim 63 wherein the guide member is electrically conductive to allow an electric current to be passed along the guide member, for actuation of the locking elements.

68. Apparatus as claimed in claim 49, wherein the carrier is flexible when in the collapsed position and adapted to be constrained by a wall of the body cavity when in the extended position.

69. Apparatus as claimed in claim 49, wherein the carrier is inflatable.

70. Apparatus as claimed in claim 69, wherein the carrier comprises an inflatable elongate balloon.

71. Apparatus as claimed in claim 70, wherein the carrier is adapted to be inserted into the opening of the body cavity in an everted position.
